(19) United States
(12) Patent Application Publication
(10) Pub. No.: US 2014/0046060 A1
(43) Pub. Date: Feb. 13, 2014

(54) METHOD OF UTILIZING RECYCLED DEUTERIUM OXIDE IN THE SYNTHESIS OF DEUTERATED COMPOUNDS

(71) Applicants: Andrew D. Jones, Needham, MA (US); Bernhard J. Paul, Arlington, MA (US)

(72) Inventors: Andrew D. Jones, Needham, MA (US); Bernhard J. Paul, Arlington, MA (US)

(73) Assignee: Concert Pharmaceuticals, Inc., Lexington, MA (US)

(21) Appl. No.: 13/820,009

(22) PCT Filed: Sep. 1, 2011
(86) PCT No.: PCT/US11/50134

§ 371 Date: Nov. 1, 2013

Related U.S. Application Data
(60) Provisional application No. 61/379,177, filed on Sep. 1, 2010.

(51) Int. Cl. C07D 473/10

(52) U.S. Cl. CPC C07D 473/10
USPC 544/271

(57) ABSTRACT

The present invention provides a method for utilizing recycled deuterium oxide synthesis of deuterated compounds.

### RELATED APPLICATION

This application claims the benefit of U.S. Provisional Application No. 61/379,177, filed on Sep. 1, 2010. The entire teachings of the above application is incorporated herein by reference.

### BACKGROUND OF THE INVENTION

In the last few years there has been an explosion of new patent applications directed to deuterated versions of existing drugs. At least three different companies have started businesses that emphasize research and development of deuterated therapeutics (Concert Pharmaceuticals, Auspex Pharmaceuticals, and Protia, LLC).

One unique aspect of synthesizing deuterated therapeutics, as opposed to their undeuterated counterparts, is the requirement for deuterated reagents and, in particular, the use of deuterated water (D₂O; deuterium oxide) as an aqueous solvent. Commercial scale production of many deuterated therapeutics requires large quantities of deuterium oxide. Because deuterium oxide is costly, it would be beneficial to find ways to recycle and re-use this reagent.

### SUMMARY OF THE INVENTION

Applicants have discovered a way to reutilize D₂O used in a synthesis reaction to perform one or more additional deuteration reactions without any detectable loss of efficiency of deuterium incorporation. This surprising discovery allows a more economical use of deuterium oxide and thus reduces the amount of deuterium oxide required for synthesis.

The invention provides a method of deuterating multiple batches of a compound of formula I:

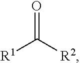

or a salt thereof, wherein:

R¹ is CH(R³)(R³);

R² is CH(R³)(R³); and

each R³ is independently H; or C₁-C₆ alkyl (i) optionally substituted with one or more cyclic groups independently selected from C₆-C₁₀ aryl, 5-10-membered heteroaryl, C₃-C₈ cycloalkyl, and 3-8-membered saturated heterocyclyl, wherein each cyclic group is optionally further substituted with one or more groups selected from C₁-C₂ alkyl, deutero-substituted C₁-C₂ alkyl and —OH; (ii) optionally substituted with one or more tautomers of the cyclic groups; and (iii) optionally substituted with deuterium, the method comprising the steps of:

(a) combining:
i. a first batch of a compound of Formula I that has previously been subjected to a first cycle of deuteration in the presence of D₂O; with
ii. D₂O and an organic solvent thereby subjecting any undeuterated or partially deuterated molecules of the compound of Formula I in the first batch to deuteration;

(b) separating the combination from step (a) into a first organic phase and a first aqueous phase;

(c) optionally combining the first organic phase with D₂O thereby subjecting any undeuterated or partially deuterated molecules of a compound of Formula I present in the first organic phase to deuteration;

(d) optionally separating the combination in step (c) into a second organic phase and a second aqueous phase;

(e) combining a second batch of a compound of Formula I with an organic solvent and the first aqueous phase thereby subjecting the compound of Formula I in the second batch to deuteration; and

(f) separating the combination in step (e) into a third organic phase and a third aqueous phase.

The method of this invention produces a compound wherein each hydrogen atom bound to the carbon atom of R¹ and R² adjacent the C═O moiety is substituted with deuterium.

### DETAILED DESCRIPTION OF THE INVENTION

### Definitions

The term “alkyl” refers to a monovalent, saturated hydrocarbon group having the indicated number or range of carbon atoms. For example, C₁-C₆ alkyl is an alkyl having from 1 to 6 carbon atoms. An alkyl may be linear or branched. Examples of alkyl groups include methyl; ethyl; propyl, including n-propyl and isopropyl; butyl, including n-butyl, isobutyl, sec-butyl, and t-butyl; pentyl, including, for example, n-pentyl, isopentyl, and neopentyl; and hexyl, including, for example, n-hexyl, and 2-methylpentyl.

The term “cycloalkyl” refers to a monovalent monocyclic or bicyclic saturated group containing only carbon ring atoms. The term “C₃-C₈ cycloalkyl” refers to a monocyclic saturated group containing between 3 and 8 carbon ring atoms. Examples of cycloalkyl include cyclopropyl, cyclobutyl, cyclopentyl, and cyclohexyl, cycloheptyl, cis- and trans-decalinyl, and norbornyl.

The term “aryl” refers to an aromatic carbocycle. The term “C₆-C₁₀ aryl” refers to a monocyclic or bicyclic, aromatic carbocycle containing between 6 and 10 ring carbon atoms. Examples of aryl are phenyl and naphthyl.

The term “saturated heterocyclyl” refers to a monovalent monocyclic or bicyclic saturated group containing between 3 and 8 ring atoms, wherein one or more ring atoms is a heteroatom independently selected from N, O, and S. Examples of saturated heterocycles include azepanyl, azetidinyl, aziridinyl, imidazolidinyl, morpholinyl, oxazolidinyl, piperazinyl, piperidinyl, pyrazolidinyl, pyrrolidinyl, tetrahydrofuranyl, and thiomorpholinyl.

The term “heteroaryl” refers to a monovalent monocyclic or bicyclic aromatic group, wherein one or more ring atoms is a heteroatom independently selected from N, O, and S. A 5-10 membered heteroaryl is a monocyclic or bicyclic heteroaryl that contains between 5 and 10 ring atoms. Examples of heteroaryl groups include furanyl, thiazolyl, isothiazolyl, isoxazolyl, oxazolyl, pyrimidinyl, pyrazolyl, pyridazinyl, pyridinyl, pyrrolyl, thiadiazolyl, thiophenyl, triazinyl, triazolyl, quinolinyl, quinazolinyl, indolyl, isoindolyl, 3,7-dihydro-1H-purine-2,6-dion-yl; xanthinyl, hypoxanthinyl, theobrominyl, uric acid, isoguaninyl, thymine, and uracilyl.

The term “substituted” refers to the replacement of one or more hydrogen atoms with the indicated substituent. For avoidance of doubt, substitutions may occur on the terminus of a moiety. For example, the terminal —CH₃ group on R³ may be substituted with one or more of the indicated substituents. “Substituted with deuterium” refers to the replacement of one or more hydrogen atoms with a corresponding number of deuterium atoms.

When a position is designated specifically as “D” or deuterium, the position is understood to have deuterium at an abundance that is at least 1000 times greater than the natural abundance of deuterium, which is 0.015% (i.e., at least 15% incorporation of deuterium). In certain embodiments, when a position is designated as “D” or deuterium that position has at least 50.1% incorporation of deuterium; at least 75% incorporation of deuterium; at least 80% incorporation of deuterium, at least 85% incorporation of deuterium; at least 90% incorporation of deuterium; at least 95% incorporation of deuterium; at least 98% incorporation of deuterium; at least 99% incorporation of deuterium; or at least 99.5% incorporation of deuterium.

When a position is designated specifically as “H” or hydrogen, the position is understood to have hydrogen at its natural isotopic abundance.

### Ketones Useful as Compounds of Formula I

In one embodiment, in the compound of Formula I, R¹ is CH₂R³; R² is CH₂R³; and each R³ is independently H; or C₁-C₆ alkyl optionally substituted with (i) one or more cyclic groups independently selected from C₆-C₁₀ aryl, 5-10-membered heteroaryl, C₃-C₈ cycloalkyl, or 3-8-membered saturated heterocyclyl, wherein each cyclic group is optionally further substituted with one or more of C₁-C₂ alkyl, deutero-substituted C₁-C₂ alkyl and —OH; (ii) one or more tautomers of the cyclic groups; and (iii) deuterium. In one aspect of this embodiment, R¹ is CH₃.

In another embodiment, in the compound of Formula I, R¹ is CH₃ and R² is —CH₂—(C₁-C₅ alkyl), wherein the C₁-C₅ alkyl is optionally substituted with (i) one or more cyclic groups independently selected from C₆-C₁₀ aryl, 5-10-membered heteroaryl, C₃-C₈ cycloalkyl, or 3-8-membered saturated heterocyclyl, wherein each cyclic group is optionally further substituted with one or more of C₁-C₂ alkyl, deutero-substituted C₁-C₂ alkyl and —OH; (ii) one or more tautomers of the cyclic groups; and (iii) deuterium. In a more specific aspect of this embodiment, R² is:

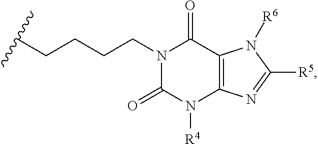

wherein R⁴ and R⁶ are independently selected from —CH₃ and —CD₃; and R⁵ is selected from H and D.

In one embodiment of the compound of formula (I), the compound is pentoxifylline:

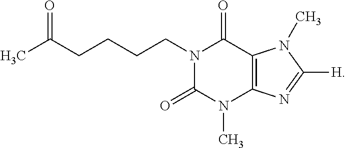

### Process Steps

The method of the present invention is useful to deuterate multiple batches of a compound of Formula I. In some embodiments, the method is useful to deuterate two batches of a compound of Formula I. In some embodiments, the method is useful to deuterate three batches of a compound of Formula I. In some embodiments, the method is useful to deuterate four or more batches of a compound of Formula I.

The amount and concentration of the compound of Formula I in each batch can vary and is only limited by the total amount of starting material (i.e. compound of Formula I) available, the amounts of the various reagents needed for deuteration available, the size of the vessel in which the deuteration reaction can take place, and the feasibility of separating the reaction mixture into an aqueous and an organic phase following a deuteration reaction. It is believed that the present invention will be particularly useful in large-scale production of deuterated compounds of Formula I because the size of the vessel in which the deuteration reaction occurs is typically the limiting factor in commercial production. This therefore requires that multiple batches of starting material be subjected to the deuteration reaction(s) in order to produce a sufficient amount of product.

In the method of this invention, the first batch of a compound of Formula I is deuterated in a multi-cycle process. The term “a first cycle of deuteration in the presence of D₂O”, when referred to the first batch, refers to a deuteration cycle in which the compound of Formula I is deuterated in the presence of D₂O having isotopic purity of at least 99% and an organic solvent. This is typically the level of purity of D₂O obtained from a commercial supplier. The product of this first cycle contains a mixture of deuterated, partially deuterated and undeuterated molecules of a compound of Formula I. The product of this first cycle partitions into the organic phase, and is typically used as the starting material in the method of this invention.

In one embodiment, the D₂O in one or more of steps (a)(ii) or (c) has at least 99% isotopic purity.

For clarity, the term “compound of Formula I” as used herein refers to undeuterated compound, wherein each hydrogen atom in the compound is present at its natural abundance. The term “partially deuterated compound of Formula I” refers to a compound, wherein at least one, but not all hydrogen atoms attached to the carbon atoms adjacent the carbonyl moiety in a compound of Formula I have been replaced with deuterium. A “fully deuterated compound of Formula I” as used herein refers to a compound wherein all hydrogen atoms attached to the carbon atoms adjacent the carbonyl moiety in a compound of Formula I have been replaced with deuterium.

A fully deuterated form obtained from the process of the invention by deuterating multiple batches of a compound of formula I,

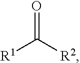

wherein R¹ is CH(R³)(R³); R² is CH(R³)(R³); and R³ is as defined hereinabove, has the formula

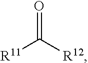

or a salt thereof, wherein:

R¹¹ is CD(R¹³)(R¹³);

R¹² is CD(R¹³)(R¹³); and

each R¹³ is independently D; or C₁-C₆ alkyl (i) optionally substituted with one or more cyclic groups independently selected from C₆-C₁₀ aryl, 5-10-membered heteroaryl, C₃-C₈ cycloalkyl, and 3-8-membered saturated heterocyclyl, wherein each cyclic group is optionally further substituted with one or more groups independently selected from C₁-C₂ alkyl, deutero-substituted C₁-C₂ alkyl and —OH; (ii) optionally substituted with one or more tautomers of the cyclic groups; and (iii) optionally substituted with deuterium,

provided that
(a) if R³ is H, then R¹³ is D, and
(b) if R³ is C₁-C₆ alkyl optionally substituted with (i), optionally substituted with (ii), and optionally substituted with (iii), then R¹³ is C₁-C₆ alkyl optionally substituted with optionally substituted with (i), optionally substituted with (ii), and optionally substituted with (iii).
   In one embodiment, R¹³ is the same group as R³. In another embodiment, R¹³ differs from R³ in having a higher extent of deuterium substitution than R³. This embodiment relates to a compound in which R³ has hydrogen atoms that exchange with deuterium under the reaction conditions.

By way of example, if the compound of Formula I is pentoxifylline:

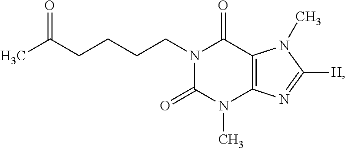

then a partially deuterated form of such compound includes:

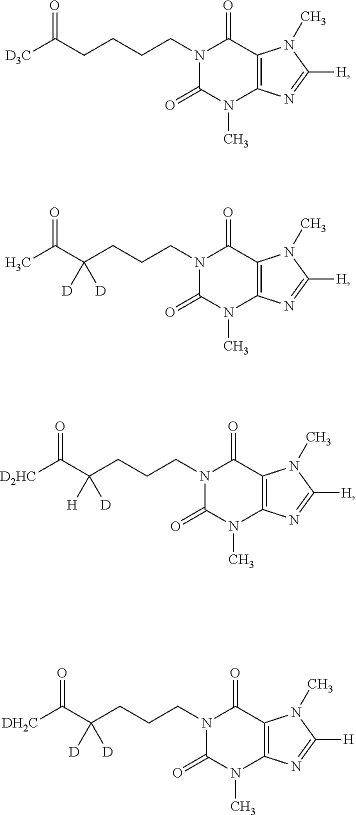

and the like. A “fully deuterated” form of pentoxifylline, as that term is used herein has the structure:

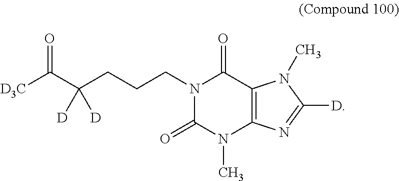

In the case of pentoxfylline the process of the present invention also deuterates the 8-position in the 3,4,5,7-tetrahydro-1H-purine-2,6-dione ring of pentoxifylline to some extent. The extent to which that position is deuterated does not in any way affect the scope or utility of the present invention.

The product of the first cycle of deuteration is subjected to a second cycle of deuteration again in the presence of D₂O having a purity of at least 99%. This increases the amount of fully deuterated product. The aqueous phase from this second cycle (“first aqueous phase”) is then isolated and is reused to deuterate a second batch of a compound of Formula I in the first cycle of deuteration of that second batch.

In certain embodiments, the first batch of a compound of Formula I is subjected to a third cycle of deuteration once again in the presence of D₂O having a purity of at least 99%. This further increases the amount of fully deuterated product. The aqueous phase from this third cycle (“second aqueous phase”) is then isolated and is reused to deuterate a different batch of a compound of Formula I in an earlier (i.e. first or second) cycle of deuteration of that different batch. In one aspect of this embodiment, the aqueous phase from the third cycle of deuteration of the first batch of compound (i.e., the second aqueous phase) is used in the second cycle of deuteration of the second batch of compound.

In certain embodiments, the second batch of compound is subjected to a third cycle of deuteration in the presence of D₂O having a purity of at least 99%. For most compounds of Formula I, three cycles of deuteration is sufficient to achieve the required levels of deuteration, typically >99%. The aqueous phase from this third cycle of deuteration of the second batch of compounds (“fifth aqueous phase”) may then be employed to deuterate another batch of compound in an earlier (i.e., first or second) cycle of deuteration.

The recycling and re-use of D₂O may be repeated a second time. Thus, in the specific aspect set forth above, the second batch of a compound of Formula I is deuterated in a second cycle with the aqueous phase from the third cycle of deuteration of the first batch of compound. Following that second cycle of deuteration of the second batch, the aqueous phase can be isolated (“fourth aqueous phase”) and used once again to deuterate a third batch of a compound of Formula I in a first cycle of deuteration.

As can be seen from the above description, any aqueous phase from a second or later cycle of deuteration can be re-used to deuterate another batch of compound in an earlier cycle of deuteration. In accordance with this, the aqueous phase from the first cycle of deuteration of any batch is not re-used.

Table 1, below shows an exemplary three-cycle deuteration on three separate batches of a compound of Formula I and indicates the deuteration agent (either ≧99% D₂O or an aqueous phase obtained from a later deuteration cycle of another batch) that may be used according to this invention.

| | Deuteration Cycle 1 | Deuteration Cycle 2 | Deuteration Cycle 3 |
|---|---|---|---|
| Batch 1 | ≧99% D₂O | ≧99% D₂O | ≧99% D₂O |
| Batch 2 | (a) Aqueous phase | Aqueous phase | ≧99% D₂O |
| | from Batch 1, | from Batch 1, | |
| | Deuteration Cycle 2 | Deuteration Cycle 3 | |
| | or | | |
| | (b) Aqueous phase | | |
| | from Batch 1, | | |
| | Deuteration Cycle 3 | | |
| Batch 3 | (a) Aqueous phase | (a) Aqueous phase | ≧99% D₂O |
| | from Batch 2, | from Batch 1, | |
| | Deuteration Cycle 2 | Deuteration Cycle 3 | |
| | or | or | |
| | (b) Aqueous phase | (b) Aqueous phase | |
| | from Batch 2, | from Batch 2, | |
| | Deuteration Cycle 3 | Deuteration Cycle 3 | |
| | or | | |
| | (c) Aqueous phase | | |
| | from Batch 1, | | |
| | Deuteration Cycle 2 | | |
| | or | | |
| | (d) Aqueous phase | | |
| | from Batch 1, | | |
| | Deuteration Cycle 3 | | |

The aqueous phase from Batch 2, Deuteration Cycle 2 that can be employed in Deuteration cycle 1 for Batch 3 is itself derived from the aqueous phase from Batch 1, Deuteration Cycle 3. Thus the ≧99% D₂O that is employed at deuteration cycle 3 for batch 1 can be reused twice. It is estimated that the recycling of D₂O (via the use of aqueous phases) in such a 3 batch, 3 cycle production provide up to a 45% reduction in the use of D₂O as compared to using fresh, ≧99% D₂O at each step. The process of this invention therefore is more economical and requires less D₂O disposal than the prior art methods.

By way of example, referring to pentoxifylline as the compound of Formula I:

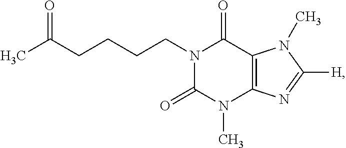

and to the structure below as the “fully deuterated” form as that term is used herein:

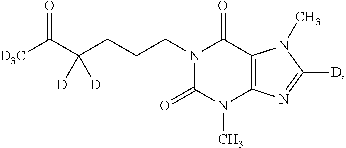

the following table shows the percentage of deuterium incorporation at each of the methyl(CO), (CO)methylene, and the imidazole ring methine carbon for successive batch runs of 50 kg of pentoxifylline in each batch:

| | | |
|---|---|---|
| | | methine |
| (CO)methylene | methyl(CO) | carbon |

Batch 1

| | | | |
|---|---|---|---|
| Exchange 1 | 88.6 | 88.8 | 13.8 |
| Exchange 2 | 97.8 | 98.0 | 26.3 |
| Exchange 3 | 99.2 | 99.2 | 37.4 |

Batch 2

| | | | |
|---|---|---|---|
| Exchange 1 (R1-2) | 82.3 | 83.8 | 6.4 |
| Exchange 2 (R1-3) | 96.7 | 96.8 | 12.4 |
| Exchange 3 | 98.9 | 98.9 | 26.4 |

Batch 3

| | | | |
|---|---|---|---|
| Exchange 1 (R2-2) | 78.0 | 80.8 | 5.8 |
| Exchange 2 (R2-2) | 95.1 | 95.6 | 11.6 |
| Exchange 3 | 98.9 | 99.0 | 27.1 |
| Exchange 4* | 99.3 | 99.4 | 32.6 |

R1-2: recycled from Batch 1, Exchange 2
R1-3: recycled from Batch 1, Exchange 3
R2-2: recycled from Batch 2, Exchange 2
R2-3: recycled from Batch 2, Exchange 3

In one embodiment, Exchange 4 is optional. In the Batch 3 run shown in the table, Exchange 4 was performed at half-volume to ensure high deuterium incorporation in Batch 3.

### EXAMPLES

### Example 1

### Deuteration of a First Batch of Pentoxifylline

A 20-L reactor equipped with a mechanical stirrer, thermocouple, and a reflux condenser was set up and purged with nitrogen. Pentoxifylline (800 g, 2.87 mol, 1.0 equiv), toluene (16 L, 20 vol), 99% D₂O (1.2 L, 1.3 kg, 66.2 mol) and K₂CO₃ (99 g, 0.72 mol, 0.25 equiv) were added to the reactor. The mixture was warmed to reflux (˜87° C.) and allowed to stir for 3-4 hours. The reaction was then cooled to 40-50° C. and the agitation stopped allowing the aqueous (“first aqueous layer”) and organic layer (“first organic layer”) to separate. The first aqueous layer was removed and discarded.

To the first organic layer was added 99.8% D₂O (1.2 L, 1.3 kg, 66.2 mol) and K₂CO₃ (99 g, 0.72 mol, 0.25 equiv). The mixture was warmed to reflux (˜87° C.) and allowed to stir for 3-4 hours. The reaction was then cooled to 40-50° C. and the agitation stopped allowing the aqueous (“second aqueous layer”) and organic layer (“second organic layer”) to separate. The second aqueous layer was removed and saved for further use.

To the second organic layer was added 99.8% D₂O (1.2 L, 1.3 kg, 66.2 mol) and K₂CO₃ (99 g, 0.72 mol, 0.25 equiv). The mixture was warmed to reflux (˜87° C.) and allowed to stir for 3-4 hours. The reaction was then cooled to 40-50° C. and the agitation stopped allowing the aqueous (“third aqueous layer”) and organic layer (“third organic layer”) to separate. The third aqueous layer was removed and saved for further use.

The third organic layer was concentrated to approximately 4.0 L and cooled to 20° C. Heptane (1600 ml, 2 vol) was added and the mixture was stirred to 30 minutes at 20° C. 27. The resultant slurry was filtered and washed with heptane (2×0.5 L). The resulting white soled was de-lumped and dried at 20-30° C. with N₂ bleed until constant weight is achieved. The resulting white solid was collected.

### Example 2

### Deuteration of a Second Batch of Pentoxifylline

A 2-L reactor equipped with a mechanical stirrer, thermocouple, and a reflux condenser was set up and purged with nitrogen. Pentoxifylline (100 g, 0.359 mol, 1.0 equiv), toluene (2 L, 20 vol), and the second aqueous layer from the first batch (150 mL, 175 g) were added to the reactor. The mixture was warmed to reflux (˜87° C.) and allowed to stir for 3-4 hours. The reaction was then cooled to 40-50° C. and the agitation stopped allowing the aqueous (“fourth aqueous layer”) and organic layer (“fourth organic layer”) to separate. The fourth aqueous layer was removed and discarded.

To the fourth organic layer was added the third aqueous layer from the first batch (150 mL, 175 g). The mixture was warmed to reflux (˜87° C.) and allowed to stir for 3-4 hours. The reaction was then cooled to 40-50° C. and the agitation stopped allowing the aqueous (“fifth aqueous layer”) and organic layer (“fifth organic layer”) to separate. The fifth aqueous layer was removed and saved for further use.

To the fifth organic layer was added 99.8% D₂O (150 mL) and K₂CO₃ (12.4 g, 0.0898 mol, 0.25 equiv). The mixture was warmed to reflux (˜87° C.) and allowed to stir for 3-4 hours. The reaction was then cooled to 40-50° C. and the agitation stopped allowing the aqueous (“sixth aqueous layer”) and organic layer (“sixth organic layer”) to separate. The sixth aqueous layer was removed and saved for further use.

To the sixth organic layer was added 99.8% D₂O (24 mL). The mixture was stirred at 40-50° C. for 30 minutes and the agitation stopped allowing the aqueous (“seventh aqueous layer”) and organic layer (“seventh organic layer”) to separate. The seventh aqueous layer was removed and may be saved for further use.

The seventh organic layer was concentrated to approximately 500 mL and cooled to 20° C. Heptane (100 ml, 1 vol) was added and the mixture was stirred to 30 minutes at 20° C. 27. The resultant slurry was filtered and washed with heptane (2×100 mL). The resulting white soled was de-lumped and dried at 20-30° C. with N₂ bleed until constant weight is achieved. The resulting white solid was collected.

What is claimed is:

1. A method of deuterating multiple batches of a compound of formula I: 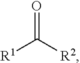
or a salt thereof, wherein:
R¹ is CH(R³)(R³);
R² is CH(R³)(R³); and
each R³ is independently H; or C₁-C₆ alkyl (i) optionally substituted with one or more cyclic groups independently selected from C₆-C₁₀ aryl, 5-10-membered heteroaryl, C₃-C₈ cycloalkyl, and 3-8-membered heterocyclyl, wherein each cyclic group is optionally further substituted with one or more independently groups selected from C₁-C₂ alkyl, deutero-substituted C₁-C₂ alkyl and —OH; (ii) optionally substituted with one or more tautomers of the cyclic groups; and (iii) optionally substituted with deuterium, the method comprising the steps of:
(a) Combining:
i. a first batch of a compound of Formula I that has previously been subjected to first cycle of deuteration in the presence of D₂O; with
ii. D₂O and an organic solvent thereby subjecting any undeuterated or partially deuterated molecules of the compound of Formula I in the first batch to deuteration;
(b) Separating the combination from step (a) into a first organic phase and a first aqueous phase;
(c) Combining a second batch of a compound of Formula I with an organic solvent and the first aqueous phase thereby subjecting the compound of Formula Ito deuteration; and
(d) Separating the combination in step (c) into a third organic phase and a third aqueous phase.

2. The method of claim 1, wherein the D₂O in step (a)(ii) has at least 99% isotopic purity.

3. The method of claim 1, further comprising the step of
(e) combining the first organic phase with D₂O thereby subjecting any undeuterated or partially deuterated molecules of a compound of Formula I present in the first organic phase to deuteration; and
(f) separating the combination in step (e) into a second organic phase and a second aqueous phase.

4. The method of claim 3, wherein the D₂O in step (e) has at least 99% isotopic purity.

5. The method of claim 1, further comprising the step of combining the third organic phase with D₂O thereby subjecting any undeuterated or partially deuterated molecules of a compound of Formula I present in the third organic phase to deuteration.

6. The method of claim 5, wherein the D₂O that is combined with the third organic phase has at least 99% isotopic purity.

7. The method of claim 1, further comprising the steps of:
(g) Combining the third organic phase with the second aqueous phase thereby subjecting any undeuterated or partially deuterated molecules of a compound of Formula I present in the third organic phase to deuteration;
(h) separating the combination from step (g) into a fourth organic phase and a fourth aqueous phase;
(i) combining the fourth organic phase with D₂O thereby subjecting any undeuterated or partially deuterated molecules of a compound of Formula I present in the fourth organic phase to deuteration; and
(j) separating the combination from step (i) into a fifth organic phase and a fifth aqueous phase.

8. The method of claim 7, wherein the D₂O in step (i) has at least 99% isotopic purity.

9. The method of claim 1, further comprising the steps of:
(k) Combining a third batch of a compound of Formula I with the fourth aqueous phase and an organic solvent thereby subjecting the compound of Formula Ito deuteration;
(l) separating the combination from step (k) into a sixth organic phase and a sixth aqueous phase; and
(m) combining the sixth organic phase with D₂O thereby subjecting any undeuterated or partially deuterated molecules of a compound of Formula I present in the sixth organic phase to deuteration.

10. The method of claim 9, wherein the D₂O in step (m) has at least 99% isotopic purity.

11. The method of claim 1, further comprising the steps of:
(k) Combining a third batch of a compound of Formula I with the fourth aqueous phase and an organic solvent thereby subjecting the compound of Formula Ito deuteration;
(l) separating the combination from step (k) into a sixth organic phase and a sixth aqueous phase;
(m) combining the sixth organic phase with the fifth aqueous phase thereby subjecting any undeuterated or partially deuterated molecules of a compound of Formula I present in the sixth organic phase to deuteration;
(n) separating the combination from step (m) into a seventh organic phase and a seventh aqueous phase; and
(o) combining the seventh organic phase with D₂O thereby subjecting any undeuterated or partially deuterated molecules of a compound of Formula I present in the seventh organic phase to deuteration.

12. The method of claim 11, wherein the D₂O in step (o) has at least 99% isotopic purity.

13. The method of claim 1, wherein in the compound of Formula I:
R¹ is CH₂R³; and
R² is CH₂R³.

14. The method of claim 13, wherein R¹ is CH₃.

15. The method of claim 14, wherein R² is —CH₂—(C₁-C₅ alkyl), wherein the C₁-C₅ alkyl is (i) optionally substituted with one or more cyclic groups independently selected from C₆-C₁₀ aryl, 5-10-membered heteroaryl, C₃-C₈ cycloalkyl, and 3-8-membered saturated heterocyclyl, wherein each cyclic group is optionally further substituted with one or more groups independently selected from C₁-C₂ alkyl, deutero-substituted C₁-C₂ alkyl and —OH; (ii) optionally substituted with one or more tautomers of the cyclic groups; and (iii) optionally substituted with deuterium.

16. The method of claim 15, wherein:
R² is: 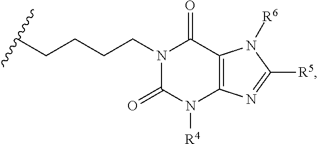
wherein R⁴ and R⁶ are independently selected from —CH₃ and —CD₃; and R⁵ is selected from H and D.

17. The method of claim 16, wherein the compound of Formula I is pentoxifylline: [chemical structure]
